(19) United States
(12) Patent Application Publication
(10) Pub. No.: US 2006/0224081 A1
(43) Pub. Date: Oct. 5, 2006

(54) Device and method for the measurement of sensory disorders with regard to various environmental conditions

(76) Inventors: Bernd Meyer, Meerbusch (DE); Dan Ziegler, Dusseldorf (DE); Michael Schweers, Meerbusch (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/565,320

(22) PCT Filed: Jul. 24, 2003
(86) PCT No.: PCT/EP03/08147

§ 371 Date: Jan. 18, 2006

(51) Int. Cl. A61B 5/00

(52) U.S. Cl. 600/555; 600/549

(57) ABSTRACT

The invention relates to a method for the measurement and/or determination of sensory disorders, in particular, related to neuropathies, whereby an air current (10) is directed at a measuring point (4) on the body of a living being, in particular a human, by means of a device (2), in order to influence the thermal sensitivity of the being at said measuring point (4), during a measuring process. The thermal sensitivity and/or a change in thermal sensitivity is correlated with a perceived temperature and, in order to determine the perceived temperature before and/or during the measurement process, at least one parameter from the environment and/or the being is recorded and analysed. The invention further relates to a device for the measurement and/or determination of sensory disorders, in particular, related to neuropathies.

The invention relates to a method and a device for measuring and/or determining sensory disorders, that is neurological disorders, especially in the case of neuropathies.

For the measurement and determination of neurological disorders and their intensities, especially in the field of neuropathic disorders of diabetic patients there are available today a variety of instruments. The spectrum of these instruments is large since the expression of these disorders involve a multiplicity of indications. The pathological picture ranges from limited effect on the sensory system to severe disorders of the short and long nerve fibers. The degree of the disorder tan be taken as a measure of the intensity.

Basically the following measurements can be carried out:

1. Qualitative measurements:
a. Reflex hammer: a test of the various reflexes and their consequences. A simple and common test based upon the motor control of the human body. The corresponding location (for example directly beneath the knee cap) is excited with the hammer and initiates in a healthy individual an extension of the leg. It is not the intensity of the response but rather the time to produce the extension which is the response determinant.
b. Descriminators like for example needles enable distinctions between the point and the stump of the needle and also distinctions between responses at different large areas of the skin to be determined. The patient is requested to respond to the contact made.
c. Monofilament (hair or bristle) for exciting receptors (tickling). With varying numbers, varying thicknesses and varying stiffnesses of hairs or bristles, the skin of the patient can be excited. Without a visual determination the patent is to indicate how these respective stimuli are felt.
d. Tiptherm to sense hot/cold. Here the characteristics of various materials to conduct heat better or to deliver heat can be used. At an end of a plastic wand, a metallic end of the same size is affixed. Alternately and randomly the tester contacts the skin of the patient with the plastic end and the metal end. The patient indicates the character of the stimulus (hot/cold)

2. Semi-Qualitative Measurement:

A tuning fork is used to determine the sensory response to oscillations (vibration). After excitation of the tuning fork, it can be placed at certain locations on the wrist or ankle. The patient is requested to indicate when the vibration with decreasing oscillation amplitude can no longer be detected. The pick-up of the mechanically/optically indicated amplitude from the turning form scale should occur within the closest possible point in time to such indication.

3. Quantitative Testing

a. Thermotest. By means of a neurothersiometer the temperature sensitivity of the patient is determined. For this purpose a temperature output device is applied to either the wrist or the back of the foot. In general the temperature of the output device is increased until the patient determines that the output device is hot or warm. In a similar way the patient indicates the point in time at which he or she can no longer feel the device from the initiation of cooling of the device. These tests are repeated and the results averaged. The process is valid and supplies good results as to the intensity or advance of any disorder which may be present.

b. Vibratest. Like with the tuning fork test a test is made using vibration intensity. The vibrations are 10 mechanically made and electronically controlled. For this purpose various intensities can be repetitively set and an exact readout afforded. Like with the thermotest, the intensity between the build up and disappearance of the vibration peak can be determined.

c. Direct current electromyography for the measurement nerve conductivity. By means of an exciter, current circuit currents are produced and applied at individual nerve fibers and detected with the tip of a probe. The elapsed time is measured. The method, although quite expensive, provides an objective indication of nerve conductivity which supplies precise results.

d. Alternating current electromyography, neurometa to the excitation current measurement correspond basically to the measurement with direct current electromyography. By contrast thereto, however, the alternating current can be set to various frequencies. This enables the separation of certain nerve fibers and provides an exact indication which can target large and small nerve fibers.

An increasingly important component of today's concern for increasing knowledge with respect to individual health is early diagnosis and the desire for qualitative and quantitative measurements at reduced cost.

In cases in which measurements are required in sufficient number to be preventative or continuous observation is required, the requisite technology is not available. It is more than understandable that not every family physician will have available an electromyograph so that it can be concluded that practically no family physician will make such an investment. In institutions and hospitals the situation is not significantly different.

The practical family physician is thus limited to the means available to him to explore whether a patient has a neuronal, for example, a neuropathic disorder or whether the state of the patient has altered in this respect. Thus for example with diabetes patients, generally long term monitoring of neuropathic disorders is required. Without the expensive and dear technology which is current available, this is practically impossible. The family physician thus can only rely on qualitative and semiqualitative measurements which allow him to determine in a yes/no manner whether the disorder may be present or there is a change in the disorder. Quantitative measurements are out of the question. Thus it is not surprising that most methods have not been validated.

Furthermore, all qualitative and most of the quantitative measurements with large and small fibers, utilize contact and vibration or contact and temperature simultaneously.

Significant error sources include, in addition, reading precision, noise or stray effects through bone conduction, the ability of the patient to concentrate and the degree of exhaustion of the patient.

The quantitative measurements especially have a weak point in the long duration required for the measurement process and the changes in the subjective perceptions of the patient.

The tuning fork test is currently in practice the most effective measurement method for the determination of neuropathic disorders of large nerve fibers.

Disorders of the small nerve fibers however require even more expensive and dear devices. The short fibers are especially important since they are subjected to neuropathic disorders first and can give an earlier indication of a severe disorder to come.

The object of the invention is to provide a simple and inexpensive method and associated device for the early detection of sensory disorders, for example neuropathic disorders, and thereby simultaneously provide the means for a family physician to ensure a quantitative and timely control or monitoring of the illness picture.

This object is achieved by a method according to claim 1 and a device according to claim 6. Preferred embodiments or features are found in the respective dependent claims.

The invention is based upon the recognition that a living creature, for example a person is able to sense even a slight air movement since the air movement appears to be significantly colder than the actual air temperature. This additional cooling effect which is found in the moving air gives rise to a sensible temperature [perceived temperature] which is less than the actual temperature of the air and is taken into consideration by the invention.

Through its heat balance, a living organism, for example the person, is closely linked with atmospheric environmental conditions. The significance of this fact to health is dependent upon the close linking of thermoregulation and circulation regulation together. An evaluation of the effect of the climate on the person is based upon the fact that the organism adjusts to the given climatic conditions. For example, in the VDI Guidelines 3787, page 2, “Method for Biometeorological valuation of Climate and Air Hygiene for City and Regional Planning”, the climate Michel model is used. It supplies an indication of the average subjective sensitivity of people.

The temperature subjectively sensed by a living organism, for example the temperature felt by a person, increases under warm, sunny and lower wind summer conditions more rapidly than the actual air temperature. In an extreme case in central Europe it can lie up to 15° C. above the actual temperature. Under pleasant, mild conditions with low wind to moderate wind, however it can drop below the actual air temperature because, for example, one must consider rapid walking and an adjustment of the clothing to the potential conditions. Under cold and especially strong wind out-door conditions, the sensed temperature drops by up to about 15° C below the actual air temperature. Sun and windless conditions can however cause the sensed temperature to increase above the actual air temperature.

By comparison to other evaluatable parameters the perceived temperature of heat sensitivity can have physiological significance.

The wind chill temperature for example a parameter used to classify cold conditions and provides a-measurement which is dependent upon wind speed, for the time required for a quarter liter of water in a plastic cylinder to freeze. Sun or compensation for the temperature by way of changes of clothing, do not enter into a determination of this parameter.

Similarly, even if to a lesser extent, on the warm side, there is the so-called discomfort index. The perceived temperature is converted into a physiologically verifiable determination of thermal sensitivity in accordance with the VDI Guidelines 3787, page 2, in the form of the so-called Predicted Mean Vote (PMV value). This value corresponds to forecast average value of the thermal estimation on a psychophysical scale of −3=cold to +3=hot.

The thermal sensitivity is based upon the conductivity and functional capacities of the short nerve fibers of the living organism, for example the person. If disorders arise with these nerve fibers, for example neuropathy disorders, the respective subjective perceptions are altered. According to the invention an altered thermal estimation is an indiction of a disorder.

The method and apparatus according to the invention are based not only on this previously described effect, but upon at least one additional parameter which has an influence on the sensed or perceived temperature like for example at least one parameter selected from air temperature, air moisture or humidity, skin temperature, skin moisture, etc.

These parameters mentioned by way of example have an effect on the perceived temperature so that according to the invention, at least one of these parameters and preferably several of them are involved in the determination of the sensed or perceived temperature.

For example, the wind chill effect is dependent upon the air moisture and temperature, that is for the same wind speed or velocity and air temperature. The subjective sensitivity of the perceived temperature will differ with differing values of the air moisture content or humidity which can be taken into consideration in the device and method of determining the sensed or perceived temperature.

Similarly, for example, evaporation cooling upon sweating can have an effect so that preferably the skin moisture and/or skin temperature are detected and the values are introduced into the determination of the sensed or perceived temperature.

According to the invention, the device can comprise at least one internal [integrated] or external sensor by means of which at least one ambient parameter and/or parameter of the living organism can be measured so that such parameter, which can have an influence on the sensed or perceived temperature can be included in the determination of the sensed or perceived temperature. Preferably a plurality of such sensors are used to allow the measurement simultaneously or successively of a plurality of for example the above mentioned parameters.

The device according to the invention, for example a mobile measuring unit, can be made optionally small to facilitate handling and thus be universally usable. Since the invention is validatable, it can be utilized as a measure of the degree of a disorder.

Preferably the method and the device can provide electronic control of the air speed with a constant spacing of measuring point or variable measuring point spacing and preferably with a constant air flow in a simple and reliable manner. Depending upon the application and the handling, based upon the air speed (speed, design and construction of the blower, sensors integrated in or connected to the system for a single environmental condition or various environmental conditions), the nature of the calculations and the determinations of the sensored temperature, the device and method can be used for determinations made on the basis of the above mentioned Michel climate model or some other known modeling approach.

In the simplest case, in a closed room, at an average room temperature and with measurements taken directly upon the skin (without covering) a disorder or the degree of a disorder can be read out or determined based upon spacing and air speed to enable a treating physician based on the read out or determined values to provide an ongoing diagnosis.

The device, simple to service, simple to use, electronically controlled, easy to calibrate or of a reliability which can be easily determined, and operates in a contactless manner, excluding noise and stray effects, while enabling multiple measurements in a rapid, validatable and inexpensive manner.

Because of its convenience and availability the device can be afforded by any family physician and reliability used. The device enables with simple means, indications of a disorder, especially neuropathic disorders or their degree, to be determined and enables diagnoses to be made earlier than has hitherto been the case and thus medications to be prescribed earlier. An earlier detection of neuropathies can be easily made and illnesses more economically and inexpensively treated. The device enables the detection of sensory, that is nerve disorders using the subjectively perceived temperature.

Two embodiments of the invention are illustrated in the drawing and are described below in greater detail.

The FIGURES show:

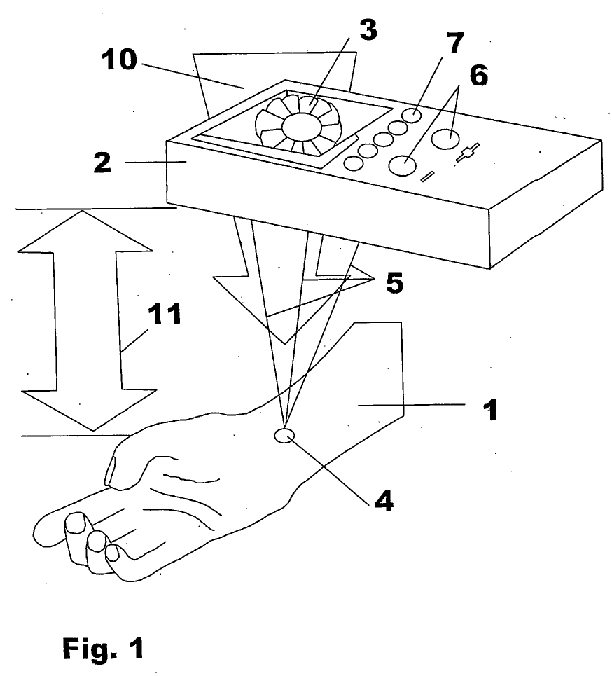

FIG. 1: a perspective view of a wrist measurement with a fixed spacing and variable air velocity; and

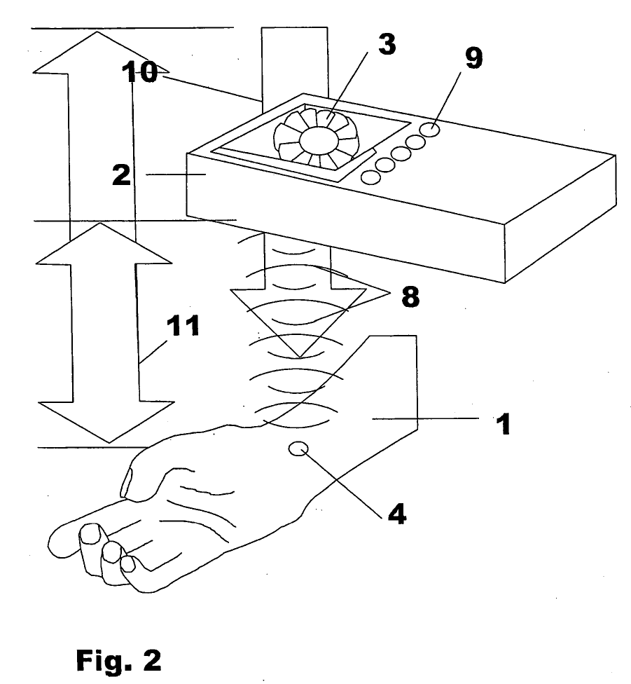

FIG. 2: a perspective view of a wrist measurement with variable spacing and constant air velocity.

A patient (1) is subjected to a measurement taken by means of a measuring device (2) in the form for example of a thenar measurement (inner wrist) on the uncovered skin (4). Alternatively, a measurement can be made at another location, for example the ankle. The body part to be subjected to measurement should preferably be stationary and protected from a draft. However this is not absolutely required because the duration of the measurement is brief. The device comprises for example a blower (3) for generating an air stream (10) preferably the measurement is taken without visual contact of the subject with the measurement device or location in order to avoid the influence of acoustic noise from the blower. In the case of a measurement with a constant spacing, the measurement device is turned on and a fixed spacing to the subject is established. This can be achieved with the aid of laser diodes whose light beams (5) intersect at the predetermined spacing of the subject from the measuring device (2). In addition the diodes can be set to have a common focus.

Preferably the use of three laser diodes which are at angular spacings of 120° from one another are used since this not only allows a fixed spacing to be reliably established but also a precise vertical positioning of the measuring device above the measuring position (4) to be set.

Then a general change of one of the parameters significant for the measurement is effected at (6), which can bring about a change in the perceived temperature, for example, the air velocity and/or the air humidity and/or the air temperature in an analog (continuous) manner or in discrete steps while the spacing is held constant. For example the air velocity can be altered by modifying the speed of the blower. Based upon the parameters set and the preferably measured parameter (by means of integrated and/or external measuring sensors) and on the basis of the climate model selected (for example the Michel Climate Model or the Wind Chill factor) the measuring device calculates the perceived temperature and displays it on a corresponding display or indicator (7). The parameters and thus the perceived temperature can be altered by the operator of the device until the patient subjectively acknowledges the air contact (the temperature change) the displayed value, for example, in terms of the perceived temperature is noted.

The parameter, for example, the speed/air humidity/air temperature is preferably altered by a further step, for example, increased and then reduced until the patient no longer senses the excitation.

These values for the perceived temperature are also noted. The notation can be effected internally of the device using a memory. An error as a result from a spurious source like for example patient stress or noise can be excluded by hysteresis measurements which are repeated. The noted values and/or their averages can provide a conclusion as to the degree of the disorder and can provide a starting point for the physician for a diagnosis. The measurement using a constant is air flow (10) requires that the measuring device (2) be switched on and moved from a greater distance toward the measuring point (4), preferably by means of a spacing measurement (8) for example with an acoustic or optical measurement. The spacing to the measuring point is displayed in an analog sense or in discrete steps on a corresponding indicator or display 9.

The spacing is reduced until the patient subjectively responds to the air flow (the temperature change).

The indicated value of the spacing and/or the perceived temperature is noted. The spacing is further shortened and then increased until the patient no longer senses the excitation.

Here as well the displayed values are noted and for example the measurement is repeated a number of times. The noted values and their averages can help provide an indication of the degree of the disorder.

The use of such a measuring device is especially not limited to hand or ankle measurements, not limited to mobile applications and not limited to human bodies. Through the measurement of environmental parameters like for example air moisture or humidity and/or air temperatures or humidity and/or the air temperature, parameter of the living organism like for example skin moisture and/or skin temperature or in general at least one parameter which influence the perceived temperature, the perceived temperature can be more precisely determined. A device with integrated or external sensors for detecting the parameters influencing the perceived temperature can according to the invention provide a more precise indication as to the perceived temperature.

1. A device for measuring and/or establishing sensory disorders especially neuropathies, characterized in that at least one device (2) directs an air stream (10) onto a measuring point (4) on the body of a living organism, especially a person to influence the thermal sensitivity during a measurement process, whereby the thermal sensitivity and/or a change in the thermal sensitivity is correlated with a perceived temperature and for determining the perceived temperature before and/or during the measurement process at least one parameter of the environment and/or the living organism is detected and evaluated.

2. The method according to claim 1 characterized in that the measurement is effected with a constant air stream (10) and a variable spacing between the device (2) and the measurement point (4).

3. The device according to claim 1 characterized in that the measurement is effected by means of a variable air stream (10) at a constant spacing between the device (2) and the measuring point (4).

4. The method according to claim 1 characterized in that the measuring point is determined optically by the device and by the superimposition of three light beams.

5. The method according to claim 1, characterized in that in the determination of the perceived temperature at least one of the parameters: ambient air temperature, air moisture content, skin temperature or skin moisture is used as an input.

6. A device for the measurement and/or determination of sensory disorders, especially neuropathies, characterized in that the device has means for producing an air stream which is directed against a measuring point on the body of the living organism and has an external or internal sensor with which at least one environmental parameter or parameter of the living organism is measurable and which is involved in the determination of a perceived temperature at the measuring point.

7. The device according to claim 6 characterized in that at least one of the parameters: air temperature, air humidity, skin temperature skin moisture is measurable by a sensor.

8. The device according to claim 3 characterized in that the air stream is variably adjustable or controllable and especially such that an air velocity can be set and/or a volume stream can be adjusted in the determination of the perceived temperature.

9. The device according to claim 3 characterized in that it includes means for determining and/or indicating and/or storing a perceived temperature.

10. The device according to claim 3 characterized in that it includes means for adjusting a desired spacing between the device and the measuring point.

11. The device according to claim 10 characterized in that the light sources comprise light emitting diodes or laser diodes whose light beams intersect at a predetermined spacing from the device.
